(19) United States
(12) Patent Application Publication
(10) Pub. No.: US 2005/0175650 A1
(43) Pub. Date: Aug. 11, 2005

(54) Composition comprising an elastomeric organopolysiloxane and additional particles

(76) Inventors: Anke Hadasch, Paris (FR); Marie-Laure Delacour, Paris (FR); Xavier Ray, Villeconin (FR)

Correspondence Address: Thomas L. Inving;FINNEGAN, HENDERSON, FARABOW, GARRETT & DUNNER, L.L.P., 1300 I Street, N.W., Washington, DC, 20005-3315, US

(21) Appl. No.: 11/014,834

(22) Filed: Dec. 20, 2004

Related U.S. Application Data
(63) Continuation-in-part of application No. 10/614,016, filed on Jul. 8, 2003.
(60) Provisional application No. 60/534,105, filed on Jan. 5, 2004.

(30) Foreign Application Priority Data
Dec. 19, 2003 (FR) 03 51125

(51) Int. Cl. A61K 7/06; A61K 7/11; A61K 7/13

(52) U.S. Cl. 424/401; 424/70.12

(57) ABSTRACT

The disclosure relates to a composition, such as a powdery cosmetic composition, comprising water, solid particles of elastomeric crosslinked organopolysiloxane and additional particles, the particles of elastomeric crosslinked organopolysiloxane and the additional particles being present in an elastomeric crosslinked organopolysiloxane/additional particles weight ratio of greater than 1.2 and less than or equal to 2.5. In one embodiment, the composition is easy to apply to keratin materials, in particular to the skin, and makes it possible to obtain a satisfactorily distributed, uniform makeup result. Further, the disclosure relates to a process for making up keratin materials comprising application of a powdery composition and a method of making the composition.

This application claims benefit of U.S. Provisional Application No. 60/534,105, filed Jan. 5, 2004. This application is also a continuation-in-part of U.S. application Ser. No.10/614,016, filed on Jul. 8, 2003, and published on Apr. 15, 2004, as U.S. patent application Publication No.2004/0071648.

The present disclosure relates to a composition, such as a powdery cosmetic composition comprising particles of elastomeric organopolysiloxane and additional particles. This disclosure further relates to a makeup or a non-therapeutic treatment process for human keratin materials, such as skin, nails, eyelashes, eyebrows, or hair. This disclosure further comprises methods of applying the disclosed powdery composition to human keratin materials and methods of making the disclosed compositions. As used herein, the term particles includes powders.

The composition of the present disclosure may be a makeup or care composition for keratin materials such as the skin. In some embodiments, it is a makeup composition.

The makeup composition may be a skin makeup product, such as a complexion product (e.g., a foundation), an eyeshadow, an eyeliner, a makeup rouge, a concealer product, a body makeup product, a lip makeup product, a nail makeup product, or a hair makeup product. In some embodiments, the composition is a skin makeup product, such as a foundation, an eyeshadow, or a makeup rouge. In other embodiments, the composition relates to a complexion makeup product, such as a foundation.

The skincare composition may be a skincare product (e.g., for the face, the body, or the hands), a skin matting product, an anti-sun product for the skin (such as the face, body, and hands), a self-tanning product, or a deodorant product.

Cosmetic compositions comprising particles of elastomeric crosslinked organic polysiloxane in aqueous medium and powders such as pigments and fillers, present in an elastomeric organopolysiloxane/powders weight ratio ranging from 0.4 to 1.2 are known from patent application WO 02/053126. These compositions have a pulverulent to pasty texture. These pasty compositions may be packaged in a dish and have a take-up surface that is elastic in nature, thus, affording a novel feel different from the feel of standard compact powders. In contrast, standard compact powders feel hard and rigid.

To use the compositions described in patent application WO 02/053126, the user gathers an amount of the composition using an applicator such as a sponge, a loop, or a brush, or alternatively using a finger, and then applies it to the keratin material to be treated or made up, for example, to the skin. However, the compositions of WO 02/053126 may be difficult to apply to the skin because of the high content of powders in those compositions and the low content of liquid. With those compositions, the user may feel a dragging sensation when spreading them, since they dry too quickly during application. As a result of this difficulty in application to the skin, the user may not be able to work the makeup product comprising this composition in order to spread it out satisfactorily and uniformly over the entire surface of the skin that the user wishes to make up. Thus, the composition becomes deposited non-uniformly on the skin. And the makeup result obtained is not homogeneous since traces of product (especially traces of color) are visible on the skin in uneven patches.

Therefore, the aim of the present disclosure, in one embodiment, is to provide a powdery composition comprising particles of elastomeric crosslinked organopolysiloxane and powders, which can be spread easily using an applicator or by finger onto keratin materials, such as the skin.

In one embodiment, the inventors have discovered that such a composition may be obtained by using a particular weight ratio between the particles of elastomeric crosslinked organopolysiloxane and the additional particles in the composition. This particular weight ratio, in some embodiments, makes it possible to improve the ease of application of the product to keratin materials such as the skin. The composition of the present disclosure, may, for example, spread easily on the skin and allows the user to satisfactorily distribute the product over the skin. Also, the present composition leads to a homogeneous makeup result, and is distributed over the skin without leaving uneven and visible traces of color.

One aspect of the present disclosure is a powdery composition comprising water, solid particles of elastomeric crosslinked organopolysiloxane, and additional particles. Here, the particles of elastomeric crosslinked organopolysiloxane and the additional particles are present in an elastomeric crosslinked organopolysiloxane/additional particles weight ratio of greater than 1.2 and less than or equal to 2.5. The composition of the present disclosure may be a cosmetic composition.

Another aspect of the disclosure is a cosmetic makeup or non-therapeutic treatment process for keratin materials such as the skin comprising application of the present composition to the keratin materials.

An additional aspect of the disclosure is a method of making a composition as described above to obtain a makeup that is easy to apply to keratin materials such as the skin. Further, the composition produces a homogeneous makeup result when distributed on the keratin materials.

The term “powdery composition” means a composition comprising a pulverulent phase and having a texture that may be in the form of a free powder, a solid composition (compact powder or pressed powder), or in the form of a pasty composition. The powdery composition is not a liquid composition (in the sense of a composition that flows under its own weight at room temperature (25° C.)). Advantageously, the powdery composition according to the disclosure is a solid composition.

The term “solid composition” means a composition that does not flow under its own weight at room temperature (25° C.) after one hour.

As described above, a composition according to the disclosure contains particles of elastomeric crosslinked organopolysiloxane. The elastomeric crosslinked organopolysiloxane may be obtained: (1) via a crosslinking addition reaction of diorganopolysiloxane comprising at least one hydrogen linked to silicon and of diorganopolysiloxane comprising ethylenically unsaturated groups linked to silicon, optionally in the presence of a platinum catalyst; (2) via a dehydrogenation crosslinking condensation reaction between a diorganopolysiloxane comprising hydroxyl end groups and a diorganopolysiloxane comprising at least one hydrogen linked to silicon, optionally in the presence of an organotin; (3) via a crosslinking condensation reaction of a diorganopolysiloxane comprising hydroxyl end groups and of a hydrolysable organopolysilane; (4) via thermal crosslinking of organopolysiloxane, optionally in the presence of an organoperoxide catalyst; or (5) via crosslinking of organopolysiloxane by high-energy radiation such as gamma rays, ultraviolet rays, or an electron beam.

In some embodiments, the elastomeric crosslinked organopolysiloxane is obtained via a crosslinking addition reaction (1) of diorganopolysiloxane comprising at least two hydrogens each linked to a silicon, and (2) of diorganopolysiloxane comprising at least two ethylenically unsaturated groups (especially vinyl groups) linked to silicon, optionally in the presence (3) of a platinum catalyst, for instance, as described in patent application EP-A-295,886.

In some embodiments, the organopolysiloxane may be obtained via reaction of dimethylpolysiloxane comprising dimethylvinylsiloxy end groups and of methyl-hydrogenopolysiloxane comprising trimethylsiloxy end groups, in the presence of a platinum catalyst.

Compound (1) is the base reagent for the formation of elastomeric organopolysiloxane, the crosslinking takes place via an addition reaction of compound (1) with compound (2) in the presence of the catalyst (3).

Compound (1) may be an organopolysiloxane comprising at least two hydrogen atoms linked to different silicon atoms in each molecule. Compound (1) may have any molecular structure, including a linear-chain or branched-chain structure or a cyclic structure. Further, compound (1) may optionally have a viscosity at 25° C. ranging from 1 to 50,000 centistokes, in order to show good miscibility with compound (2).

The organic groups linked to the silicon atoms in compound (1) may be alkyl groups such as methyl, ethyl, propyl, butyl, or octyl; substituted alkyl groups such as 2-phenylethyl, 2-phenylpropyl, or 3,3,3-trifluoropropyl; aryl groups such as phenyl, tolyl, or xylyl; substituted aryl groups such as phenylethyl; and substituted monovalent hydrocarbon-based groups such as an epoxy group, a carboxylate ester group, or a mercapto group.

Compound (1) may thus be chosen from methylhydrogenopolysiloxanes comprising trimethylsiloxy end groups, dimethylsiloxane-methylhydrogenosiloxane copolymers comprising trimethylsiloxy end groups, and dimethylsiloxane-methylhydrogenosiloxane cyclic copolymers.

Compound (2) is advantageously a diorganopolysiloxane comprising at least two lower alkenyl groups (for example C₂-C₄) and the lower alkenyl groups may be chosen from vinyl, allyl, and propenyl groups. These lower alkenyl groups may be located in any position of the organopolysiloxane molecule, but are optionally located at the end of the organopolysiloxane molecule. The organopolysiloxane which is compound (2) may have a branched-chain, linear-chain, cyclic, or network structure. In certain embodiments, compound (2) has a linear-chain structure. Compound (2) may have a viscosity ranging from the liquid state to the gum state. And in some embodiments, compound (2) has a viscosity of at least 100 centistokes at 25° C.

The organopolysiloxanes (2) may be chosen from methylvinylsiloxanes, methylvinylsiloxane-dimethylsiloxane copolymers, dimethylpolysiloxanes comprising dimethylvinylsiloxy end groups, dimethylsiloxane-methylphenylsiloxane copolymers comprising dimethylvinylsiloxy end groups, dimethylsiloxane-diphenylsiloxane-methylvinylsiloxane copolymers comprising dimethylvinylsiloxy end groups, dimethyl-siloxane-methylvinylsiloxane copolymers comprising trimethylsiloxy end groups, dimethylsiloxane-methylphenylsiloxane-methylvinylsiloxane copolymers comprising trimethylsiloxy end groups, methyl(3,3,3-trifluoropropyl)polysiloxane comprising dimethylvinylsiloxy end groups, and dimethylsiloxane-methyl(3,3,3-trifluoropropyl)siloxane copolymers comprising dimethylvinylsiloxy end groups.

Besides the above-mentioned alkenyl groups, the other organic groups that may be linked to the silicon atoms in compound (2) include alkyl groups such as methyl, ethyl, propyl, butyl, or octyl; substituted alkyl groups such as 2-phenylethyl, 2-phenylpropyl or 3,3,3-trifluoropropyl; aryl groups such as phenyl, tolyl, or xylyl; substituted aryl groups such as phenylethyl; and substituted monovalent hydrocarbon-based groups such as an epoxy group, a carboxylate ester group, or a mercapto group.

In some embodiments, the elastomeric organopolysiloxane may be obtained via reaction of dimethylpolysiloxane comprising dimethylvinylsiloxy end groups and of methylhydrogenopolysiloxane comprising trimethylsiloxy end groups, in the presence of a platinum catalyst.

Advantageously, in some embodiments, the sum of the number of ethylenic groups per molecule of compound (2) and of the number of hydrogen atoms linked to silicon atoms per molecule of compound (1) is 5 or greater.

It may also be advantageous (but not required) for compound (1) to be added in an amount such that the molecular ratio between the total amount of hydrogen atoms linked to silicon atoms in compound (1) and the total amount of all the ethylenically unsaturated groups in compound (2) is within the range from 1.5/1 to 20/1.

Compound (3) may be the catalyst for the crosslinking reaction, and may be chosen from chloroplatinic acid, chloroplatinic acid-olefin complexes, chloroplatinic acid-alkenylsiloxane complexes, chloroplatinic acid-diketone complexes, platinum black, or platinum on a support. The catalyst (3) may be added in a proportion of from 0.1 to 1,000 parts by weight and in particular embodiments from 1 to 100 parts by weight, as clean platinum metal per 1,000 parts by weight of the total amount of compounds (1) and (2).

Such particles of elastomeric crosslinked organopolysiloxane are described in patent applications JP-A-61-194,009, EP-A-242,219, and EP-A-381,166.

Advantageously, in some embodiments, the elastomer is a non-emulsifying elastomer. The term “non-emulsifying” defines organopolysiloxane elastomers comprising no polyoxyalkylene units.

Elastomers in powder form that may be used in accordance with the present disclosure include those sold under the names “DC 9505” and “DC 9506” by the company Dow Corning.

According to one embodiment of the disclosure, the particles of elastomeric crosslinked organopolysiloxane used may be in the form of an aqueous dispersion.

The elastomeric organopolysiloxanes of the present disclosure may be chosen from the crosslinked polymers described in patent application JP-A-10/175,816 or patent U.S. Pat. No. 5,928,660. According to this patent application, they are obtained via a crosslinking addition reaction, in the presence of a catalyst such as those of the platinum type, of at least:
(a) one organopolysiloxane (i) comprising at least two vinyl groups in a-m position on the silicone chain per molecule; and
(b) one organosiloxane (ii) comprising at least one hydrogen atom linked to a silicon atom per molecule.

In some embodiments, the organopolysiloxane (i) is chosen from polydimethylsiloxanes such as an α-ω-dimethylvinyl polydimethylsiloxane.

The elastomeric organopolysiloxanes that are present in the composition according to the disclosure may be in the form of an aqueous suspension. This suspension may be obtained by the following steps:
(a) mixing an organopolysiloxane (i) comprising at least two vinyl groups in α-ω position on the silicone chain per molecule and an organosiloxane (ii) comprising at least one hydrogen atom linked to a silicon atom per molecule;
(b) adding a catalyst, such as a catalyst of platinum type;
(c) adding an aqueous phase comprising an emulsifier to form an emulsion; and
(d) performing emulsion polymerization of the organopolysiloxane (i) and the organosiloxane (ii) in the presence of a platinum catalyst.

The emulsifier may be chosen from nonionic, cationic, and anionic surfactants with an HLB≧8. The emulsifier may be one or more nonionic surfactants.

The proportion of surfactants used in accordance with the method above, may be from 0.1 to 20 parts by weight per 100 parts by weight of the elastomeric organopolysiloxane composition. In some embodiments, the proportion of surfactants may be from 0.5 to 10 parts by weight per 100 parts by weight of the elastomeric organopolysiloxane composition.

After step (c), it is possible to dry the particles obtained, i.e., to evaporate all or some of the trapped water from the particles.

The organopolysiloxanes of the present disclosure may be in the form of deformable solid particles having a certain hardness, which may be measured using a Shore A durometer (according to ASTM standard D2240) at room temperature or via Japanese method JIS-A. This hardness may be measured on an elastomeric block prepared for this purpose as follows: mixing of the organopolysiloxane (i) and of the organosiloxane (ii); removing air from the mixture; molding and vulcanization the mixture in an oven at 100° C. for 30 minutes; and cooling the mixture to room temperature, followed by measuring the hardness. The density of the mixture is also determined on this block of elastomer.

The organopolysiloxane may have a JIS-A hardness of less than or equal to 80. In particular embodiments of the present disclosure, the organopolysiloxane may have a JIS-A hardness ranging from 10 to 80, less than or equal to 65, and from 15 to 65.

For organopolysiloxane particles that are dispersed in water, it is possible to use those sold under the names BY 29-122 and BY 29-119 by the company Dow Corning.

The particles of elastomeric crosslinked organopolysiloxane may be present in the present composition in an amount ranging from 20% to 50% by weight relative to the total weight of the composition, and in certain embodiments, from 30% to 50% by weight relative to the total weight of the composition. Further, in some embodiments, the particles are present in an amount ranging from 35% to 45% by weight or from 35% to 40% by weight relative to the total weight of the composition.

Further, the composition according to the disclosure contains at least some additional particles different from the particles of elastomeric crosslinked organo-polysiloxane described previously.

The additional particles may be chosen from pulverulent dyestuffs, fillers and fibers, and mixtures thereof. The composition according to the disclosure may thus comprise dyestuffs and/or fillers and/or fibers.

The pulverulent dyestuff may, in certain embodiments of the present disclosure, be chosen from pigments and nacres, and mixtures thereof.

The term “pigments” as used herein should be understood as meaning white or colored, mineral or organic particles of any form, which are insoluble in the physiological medium, and are intended to color the composition.

The term “nacres” as used herein should be understood as meaning iridescent particles of any form, such as those produced by certain molluscs in their shell or alternatively synthesized.

The pigments may be white or colored and mineral and/or organic. Among the mineral pigments that may be mentioned are titanium dioxide (optionally surface-treated), zirconium oxides, or cerium oxides, and also zinc oxides, iron oxides (black, yellow, or red) or chromium oxides, manganese violet, ultramarine blue, chromium hydrate and ferric blue, and metal powders (such as aluminium powder and copper powder).

Among the organic pigments that may be mentioned are carbon black, pigments of D&C type, and lakes based on cochineal carmine, barium, strontium, calcium, or aluminium.

The nacreous pigments may be chosen from white nacreous pigments such as mica coated with titanium or with bismuth oxychloride, colored nacreous pigments such as titanium mica with iron oxides, titanium mica with, optionally, ferric blue or chromium oxide, titanium mica with an organic pigment of the above-mentioned type, and also nacreous pigments based on bismuth oxychloride.

The pulverulent dyestuffs may be present in the composition according to the disclosure in an amount ranging from 0.5% to 50% by weight and, in some embodiments, from 1 % to 30% by weight, relative to the total weight of the composition.

Additionally, the composition according to the disclosure may comprise fillers. The term “fillers” as used herein should be understood as meaning colorless or white, mineral or synthetic particles of any form, which are insoluble in the medium of the composition, irrespective of the temperature at which the disclosed composition is manufactured. The fillers may be mineral or organic, and of any form including platelet-form, spherical, or oblong, regardless of the crystallographic form (which may be, for example, laminar, cubic, hexagonal, orthorhombic, etc.). Fillers that may be used in the present composition include talc, mica, silica, kaolin, polyamide (Nylon®) powder, poly-β-alanine powder and polyethylene powder, tetrafluoroethylene polymer (Teflon®)) powders, lauroyllysine, starch, boron nitride, hollow polymer microspheres such as those of polyvinylidene chloride/acrylonitrile, for instance Expancel® (Nobel Industrie) or of acrylic acid copolymers, ethylene glycol dimethacrylate and lauryl methacrylate copolymer powders, such as those sold under the name Polytrap® 6603 Adsorber by the company RP Scherer, silicone resin microbeads (e.g., Tospearls® from Toshiba), precipitated calcium carbonate, magnesium carbonate and magnesium hydrocarbonate, hydroxyapatite, hollow silica microspheres, glass or ceramic microcapsules, metal soaps derived from organic carboxylic acids comprising from 8 to 22 carbon atoms and in some embodiments from 12 to 18 carbon atoms, for example, zinc stearate, magnesium stearate or lithium stearate, zinc laurate, or magnesium myristate.

In particular embodiments, the fillers are chosen from mica, polyamide powders, ethylene glycol dimethacrylate and lauryl methacrylate copolymer powders, and boron nitride, and mixtures thereof.

And the fillers may be present in the composition in an amount ranging from 0.5% to 40% by weight, and in some embodiments, from 1 % to 35% by weight, relative to the total weight of the composition. In particular embodiments of the composition, the fillers may be present in an amount ranging from 5% to 30% by weight or from 15% to 30% by weight, relative to the total weight of the composition.

The composition according to the disclosure may also comprise fibers.

The term “fiber” as used herein should be understood as meaning an object of length L and diameter D such that L is very much greater than D, and D is the diameter of the circle in which the cross section of the fiber is inscribed. In some embodiments that contain fibers, the ratio L/D (or shape factor) range from 3.5 to 2500, from 1.5 to 2500, from 5 to 500, from 3.5 to 500, or from 5 to 150.

The fibers that may be used in the composition of the disclosure may be mineral or organic fibers of synthetic or natural origin. They may be short or long, individual or organized (for example, braided), hollow or solid. Further, they may have any shape, and may have a circular or polygonal (square, hexagonal, or octagonal) cross section, depending on the intended application for the composition comprising the fibers. The ends of the fibers may be blunt and/or polished to prevent injury to the surface of the keratin materials to which the composition is applied.

In some embodiments, the fibers have a length (L) ranging from 1 μm to 10 mm, from 0.1 mm to 5 mm, and from 0.3 mm to 1 mm. Further, their cross-section may be within a circle of diameter ranging from 2 nm to 500 μm, from 100 nm to 100 μm, or from 1 μm to 50 μm. The weight or yarn count of the fibers is often given in denier or decitex, and represents the weight in grams per 9 km of yarn. In some embodiments, the fibers present in the composition of the present disclosure have a yarn count that ranges from 0.01 to 10 denier, from 0.1 to 2 denier, or from 0.3 to 0.7 denier.

The fibers can be those used in the manufacture of textiles, such as silk fiber, cotton fiber, wool fiber, flax fiber, and cellulose fiber extracted, by way of example, from wood, from plants, or from algae, rayon fiber, polyamide (Nylon®) fiber, viscose fiber, acetate fiber, in particular rayon acetate fiber, poly(p-phenyleneterephthalamide) (or aramide) fiber, in particular Kevlar® fiber, acrylic polymer fiber, such as polymethyl methacrylate fiber or poly(2-hydroxyethyl methacrylate) fiber, polyolefin fiber and polyethylene or polypropylene fiber, glass fiber, silica fiber, carbon fiber, such as carbon fiber in graphite form, polytetrafluoroethylene (such as Teflon®) fiber, insoluble collagen fiber, polyester fiber, polyvinyl chloride fiber or polyvinylidene chloride fiber, polyvinyl alcohol fiber, polyacrylonitrile fiber, chitosan fiber, polyurethane fiber, polyethylene phthalate fiber, and fibers formed from a mixture of polymers, such as those mentioned above. In some embodiments, the mixture of polymers may comprise polyamide/polyester fibers. In particular embodiments, the fibers are polyamide (Nylon®) fibers.

Fibres used in surgery may also be used in the present composition, for instance, the resorbable synthetic fibers prepared from glycolic acid and caprolactone (Monocryl® from Johnson & Johnson); resorbable synthetic fibers comprising copolymer of lactic acid and glycolic acid (Vicryl® from Johnson & Johnson); polyterephthalic ester fibers (Ethibond® from Johnson & Johnson); and stainless steel threads (Acier® from Johnson & Johnson).

Moreover, the fibers may be treated or untreated at the surface, and coated or uncoated with a protective layer. As coated fibers that may be used in the composition, mention may be made of polyamide fibers coated with copper sulphide to give an anti-static effect (for example R-STAT® from Rhodia) or another polymer allowing a special organization of fibers (treatment of the specific surface). Mention may also be made of fibers coated with mineral or organic pigments, such as the pigments described below. In some embodiments, fibers of synthetic origin and organic fibers, such as those used in surgery, are used.

The fibers that may be used in the composition according to the present disclosure may be polyamide fibers, cellulose fibers, poly(p-phenyleneterephthalamide) fibers, or polyethylene fibers. Their length (L) may optionally range from 0.1 mm to 5 mm, from 0.25 mm to 1.6 mm, and from 1 μm to 50 μm. In certain embodiments, the polyamide fibers used are those sold by Etablissements P. Bonte under the name “Polyamide 0.9 Dtex 3 mm®,” and have a mean diameter of 6 μm, a yarn count of about 0.9 dtex, and a length (L) ranging from 0.3 mm to 5 mm, may be used. In additional embodiments, polyamide fibers sold under the name of Fiberlon 931-D1-S by the company LCW, having a yarn count of about 0.9 dtex, and a length of about 0.3 mm may be used in the composition. Cellulose (or rayon) fibers with a mean diameter of 50 μm and a length ranging from 0.5 mm to 6 mm may also be used, for instance those sold under the name “Natural rayon flock fiber RC1BE-N003-M04®” by the company Claremont Flock. Polyethylene fibers, for instance those sold under the name “Shurt Stuff 13,099 F®” by the company Mini Fibers, may also be used.

The composition according to the disclosure may also comprise “rigid” fibers, as opposed to the fibers mentioned above, which are not rigid fibers. The rigid fibers, which are initially substantially straight when placed in a dispersing medium, do not undergo a substantial change in shape (which is reflected by the angular condition defined below) and in fact keep a shape in the dispersing medium that may be described as substantially straight and linear. The angle condition parameter described below reflects the stiffness of the fibers. It is difficult to express fiber stiffness and rigidity by another parameter for objects that are as small as the rigid fibers described in the present disclosure.

The stiffness of the fibers is reflected by the following angular condition: in some embodiments, the fibers have an angle condition of at least 50% numerically. This means that the angle formed between the tangent to the longitudinal central axis of the fiber and the straight line connecting the said end to the point on the longitudinal central axis of the fiber corresponding to half the length of the fiber is less than 15°, and the angle formed between the tangent to the longitudinal central axis of the fiber at a point half-way along the fiber and the straight line connecting one of the ends to the point on the longitudinal central axis of the fiber (corresponding to half the length of the fiber), is less than or equal to 15° for the same fiber length ranging from 0.8 mm to 5 mm. In some embodiments, the fiber length can range from 1 mm to 4 mm, from 1 mm to 3 mm, or could be approximately 2 mm. In other embodiments, the fibers have an angle condition of at least 75% or at least 90% numerically.

Advantageously, in some embodiments the angle mentioned above is measured at the two ends of the fiber and at a point half way along the fiber. In other words, three measurements are taken in this case, and the average of the measured angles is less than or equal to 15°.

This means that the tangent, at any point on the fiber, forms an angle of less than 15°.

In the present disclosure, the angle formed by the tangent at a point on the fiber is the angle formed between the tangent to the longitudinal central axis of the fiber at the said point on the fiber and the straight line connecting the end of the fiber that is closest from the said point to the point on the longitudinal central axis of the fiber corresponding to half the length of the fiber.

Generally, the rigid fibers that may be used in the composition according to the disclosure have the same or substantially the same fiber length.

More specifically, when a medium in which the rigid fibers are dispersed is observed, a fiber concentration of 1% by weight is measured by a microscope with an objective lens allowing a magnification of 2.5 and with full-field vision. Additionally, a numerical majority of the rigid fibers, i.e., at least 50% numerically of the rigid fibers, must satisfy the angular condition defined above. In some embodiments, at least 75% numerically of the rigid fibers or at least 90% numerically of the rigid fibers must satisfy the angular condition defined above. The measurement leading to the angle value is performed for the same length of fibers, this length being in the range from 0.8 mm to 5 mm, from 1 to 4 mm, from 1 to 3 mm, or approximately 2 mm, as described above.

The medium in which the observation described above is performed is a dispersing medium that ensures good dispersion of the rigid fibers, for example, water or an aqueous gel of clay or of associative polyurethane. A direct observation of the composition comprising the rigid fibers may even be performed. And a sample of the prepared composition or dispersion may be placed between a slide and cover slip for observation by microscope with an objective lens allowing a magnification of 2.5 and the microscope must allow full-field vision. Full-field vision allows the fiber lengths to be viewed in their entirety.

The rigid fibers that may be used in the composition may be chosen from fibers of a synthetic polymer, including polyesters, polyurethanes, acrylic polymers, polyolefins, polyamides, such as non-aromatic polyamides, and aromatic polyimideamides.

Examples of rigid fibers that may be used include:
polyester fibers, such as those obtained by chopping yarns sold under the names Fiber 255-100-R11-242T Taille 3 mm (eight-lobed cross section), Fiber 265-34-R11-56T Taille 3 mm (round cross section), and Fiber Coolmax 50-34-591 Taille 3 mm (four-lobed cross section) by the company Dupont de Nemours;
polyamide fibers, such as those sold under the names Trilobal Nylon 0.120-1.8 DPF; Trilobal Nylon 0.120-18 DPF; and Nylon 0.120-6 DPF by the company Cellusuede Products; or obtained by chopping yarns sold under the name Fiber Nomex Brand 430 Taille 3 mm by the company DuPont de Nemours;
polyimideamide fibers, such as those sold under the names “Kermel” and “Kermel Tech” by the company Rhodia;
poly(p-phenyleneterephthalamide) (or aramide) sold especially under the name Kevlar® by the company Dupont de Nemours; and
fibers with a multilayer structure comprising alternating layers of polymers chosen from polyesters, acrylic polymers, and polyamides, such as those described in documents EP-A-6,921,217, EP-A-686,858 and U.S. Pat. No. 5,472,798. Such fibers are sold under the names “Morphotex” and “Teijin Tetron Morphotex” by the company Teijin.

Rigid fibers that may be used in certain embodiments include aromatic polyimideamide fibers. Polyimideamide yarns or fibers that may be used for the composition of the disclosure are described, for example, in the document from R. Pigeon and P. Allard, Chimie Macromoléculaire Appliquée, 40/41 (1974), pp.139-158 (No. 600), and in U.S. Pat. No. 3,802,841, FR-A-2,079,785, EP-A1-0,360,728, and EP-A-0,549,494.

The aromatic polyimideamide fibers include polyimideamide fibers comprising repeating units of formula: 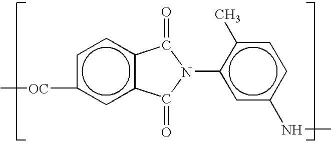
obtained by polycondensation of tolylene diisocyanate and trimellitic anhydride.

These fibers may be present in the composition in an amount ranging from 0.5% to 20% by weight, from 0.5% to 15% by weight, from 0.5% to 8%, or from 2% to 8% by weight, relative to the total weight of the composition.

Advantageously, the composition may comprise a total content of additional particles (including dyestuff, fillers, and fibers) in an amount ranging from 20% to 50% by weight, from 25% to 45% by weight, from 25% to 40% by weight, or from 30% to 40% by weight, relative to the total weight of the composition.

Optionally, the particles of elastomeric crosslinked organopolysiloxane and the additional particles are present in the composition such that the particles of crosslinked organopolysiloxane/additional particles weight ratio ranges from 1.21 to 2, from 1.21 to 1.5, or from 1.21 to 1.3.

The composition according to the invention may comprise water. The water may be a floral water such as cornflower water and/or a mineral water such as eau de Vittel, eau de Lucas, or eau de La Roche Posay, and/or a spring water. And the water content in the composition may range from 5% to 30% by weight, from 10% to 25% by weight, from 15% to 25% by weight, or from 20% to 25% by weight, relative to the total weight of the composition.

The composition may also comprise a polyol that comprises from 2 to 20 carbon atoms. In some embodiments, the composition may comprise polyols from 2 to 10 carbon atoms or from 2 to 6 carbon atoms. Polyols that may be mentioned include glycerol, propylene glycol, butylene glycol, pentylene glycol, hexylene glycol, dipropylene glycol, and diethylene glycol, and mixtures thereof.

The composition may comprise a polyol in an amount ranging from 1% to 20%, such as from 3% to 15% by weight, relative to the total weight of the composition.

The composition may contain other usual cosmetic ingredients, which may be chosen from antioxidants, fragrances, preserving agents, neutralizers, surfactants, waxes, oils, sunscreens, vitamins, moisturizers, self-tanning compounds, and anti-wrinkle active agents.

A person skilled in the art will take care to select these optional additional compound(s), and the amounts thereof, such that the advantageous properties of the composition according to the disclosure are not, or are not substantially, adversely affected by the envisaged addition.

Advantageously, the composition according to the disclosure may have a hardness ranging from 0.07 N to 0.4 N or from 0.1 to 0.35 N, and an elasticity (EL) ranging from 15% to 80% or from 30% to 70%.

The hardness and the elasticity of a product comprising the composition are measured at 20° C. using the texturometer sold under the name TA-XT2i by the company Rheo, which is equipped with a stainless-steel spindle in the form of a bead 12.7 mm in diameter, by measuring the change in force (compression force or stretching force) (F) as a function of time, during the following operation: The spindle is displaced at a speed of 0.1 mm/s and then penetrates into the product to a penetration depth of 0.3 mm. When the spindle has penetrated into the product to a depth of 0.3 mm, the spindle is removed at a speed of 0.1 mm/s. During the withdrawal of the spindle, the force (compression force) decreases greatly until it becomes 0 after a time (t). During the operation, the spindle effects a to-and-fro motion within 6 seconds.

The hardness corresponds to the maximum compression force measured during the operation and is expressed in Newtons.

The elasticity EL, expressed as a percentage, is determined via the relationship:
EL(%)=100×(t−3)/(6−3)

The composition may be prepared by mixing together the various ingredients in a Baker-Perkins turbomixer/granulator or in a continuous twin-screw blender such as the BC21 extruder-blender from the company Clextral.

The composition may be packed in a dish or a case by pressing the mixture of ingredients.

The composition of the present disclosure is illustrated in greater detail by the examples below. Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

### EXAMPLE 1

A complexion makeup product having the composition below was prepared:

| | | |
|---|---|---|
| Particles of crosslinked | 59 | g |
| polydimethylsiloxane as an aqueous | (37.1 | g AM*) |
| dispersion containing 63% by | | |
| weight of crosslinked polymer | | |
| (BY 29-119 from Dow Corning) | | |
| Glycerol | 5 | g |
| Propylene glycol | 5 | g |
| Preserving agents | 1 | g |
| Nylon powder (Orgasol ® 2002 | 10 | g |
| Extra D Nat Cos from Atofina) | | |
| Ethylene glycol dimethacrylate | 5 | g |
| and lauryl methacrylate | | |
| copolymer powder (Polytrap ® | | |
| 6603 Adsorber sold by RP | | |
| Scherer) | | |
| 0.9 Dtex polyamide fibers 0.3 | 5 | g |
| mm long (from Paul Bonte) | | |
| Pigments (iron oxides, | 10 | g |
| titanium dioxide) | | |

*Active Material (AM)

After mixing together the ingredients, the product was packaged in a-dish by pressing. The product was easily applied to the skin and a uniformly distributed makeup result was obtained.

The product obtained had a hardness of 0.2 N and an elasticity of 55%, and these parameters were measured according to the conditions described previously.

### EXAMPLE 2

A skin matting product having the composition below was prepared:

| | | |
|---|---|---|
| Particles of crosslinked | 59 | g |
| polydimethylsiloxane as an aqueous | (37.1 | g AM*) |
| dispersion containing 63% by | | |
| weight of crosslinked polymer | | |
| (BY 29-119 from Dow Corning) | | |
| Glycerol | 5 | g |
| Propylene glycol | 5 | g |
| Preserving agents | 1 | g |
| Nylon powder (Orgasol ® 2002 | 20 | g |
| Extra D Nat Cos from Atofina) | | |
| Ethylene glycol dimethacrylate | 5 | g |
| and lauryl methacrylate | | |
| copolymer powder (Polytrap ® | | |
| 6603 Adsorber by RP Scherer) | | |
| 0.9 Dtex polyamide fibers 0.3 | 5 | g |
| mm long (from Paul Bonte) | | |

*Active Material (AM)

After mixing together the ingredients, the product was packaged in a dish by pressing. The product was easy to apply to the skin.

### EXAMPLE 3

A complexion makeup product having the composition below was prepared:

| | | |
|---|---|---|
| Particles of crosslinked | 59 | g |
| polydimethylsiloxane as an aqueous | (37.1 | g AM*) |
| dispersion containing 63% by | | |
| weight of crosslinked polymer | | |
| (BY 29-119 from Dow Corning) | | |
| Glycerol | 5 | g |
| Propylene glycol | 5 | g |
| Preserving agents | 1 | g |
| Nylon powder (Orgasol ® 2002 | 15 | g |
| Extra D Nat Cos from Atofina) | | |
| Ethylene glycol dimethacrylate | 5 | g |
| and lauryl methacrylate | | |
| copolymer powder (Polytrap ® | | |
| 6603 Adsorber sold by RP | | |
| Scherer) | | |
| Pigments (iron oxides, | 10 | g |
| titanium dioxide) | | |

*Active Material (AM)

After mixing together the ingredients, the product was packaged in a dish by pressing. The product was easy to apply to the skin, and allowed uniform distribution of the makeup product on the skin.

### EXAMPLE 4

A skin matting product having the composition below was prepared:

| | | |
|---|---|---|
| Particles of crosslinked | 59 | g |
| polydimethylsiloxane as an aqueous | (37.1 | g AM*) |
| dispersion containing 63% by | | |
| weight of crosslinked polymer | | |
| (BY 29-119 from Dow Corning) | | |
| Glycerol | 5 | g |
| Propylene glycol | 5 | g |
| Preserving agents | 1 | g |
| Nylon powder (Orgasol ® 2002 | 25 | g |
| Extra D Nat Cos from Atofina) | | |
| Ethylene glycol dimethacrylate | 5 | g |
| and lauryl methacrylate | | |
| copolymer powder (Polytrap ® | | |
| 6603 Adsorber sold by RP | | |
| Scherer) | | |

*Active Material (AM)

After mixing together the ingredients, the product was packaged in a dish by pressing. The product was easy to apply to the skin.

### EXAMPLE 5

A complexion makeup product having the composition below was prepared:

| | | |
|---|---|---|
| Particles of crosslinked | 59 | g |
| polydimethylsiloxane as an aqueous | (37.1 | g AM*) |
| dispersion containing 63% by | | |
| weight of crosslinked polymer | | |
| (BY 29-119 from Dow Corning) | | |
| Glycerol | 5 | g |
| Propylene glycol | 5 | g |
| Preserving agents | 1 | g |
| Nylon powder (Orgasol ® 2002 | 10 | g |
| Extra D Nat Cos from Atofina) | | |
| Pigments (iron oxides, | 20 | g |
| titanium dioxide) | | |

*Active Material (AM)

After mixing together the ingredients, the product was packaged in a dish by pressing. The product was easy to apply to the skin and allowed for uniform distribution of the makeup product on the skin.

### EXAMPLE 6

A skin matting product having the composition below was prepared:

| | | |
|---|---|---|
| Particles of crosslinked | 59 | g |
| polydimethylsiloxane as an aqueous | (37.1 | g AM*) |
| dispersion containing 63% by | | |
| weight of crosslinked polymer | | |
| (BY 29-119 from Dow Corning) | | |
| Glycerol | 5 | g |
| Propylene glycol | 5 | g |
| Preserving agents | 1 | g |
| Nylon powder (Orgasol ® 2002 Extra | 30 | g |
| D Nat Cos from Atofina) | | |

*Active Material (AM)

After mixing together the ingredients, the product was packaged in a dish by pressing. The product was easy to apply to the skin.

### EXAMPLE 7

An eyeshadow product having the composition below was prepared:

| | | |
|---|---|---|
| Crosslinked polydimethylsiloxane | 42 | g |
| particles (DC 9506 from Dow Corning) | | |
| Water | 15 | g |
| Glycerol | 10 | g |
| Preserving agents | 1 | g |
| Ethylene glycol dimethacrylate | 2 | g |
| and lauryl methacrylate | | |
| copolymer powder (Polytrap ® | | |
| 6603 Adsorber sold by RP | | |
| Scherer) | | |
| Nacres | 30 | g |

After mixing together the ingredients, the product was packaged in a dish by pressing. The product was easy to apply to the skin.

### EXAMPLE 8

A complexion makeup product having the composition below was prepared:

| | | |
|---|---|---|
| Crosslinked polydimethylsiloxane | 40 | g |
| particles (DC 9506 from | | |
| Dow Corning) | | |
| Water | 19 | g |
| Glycerol | 5 | g |
| Propylene glycol | 5 | g |
| Preserving agents | 1 | g |
| Nylon powder (Orgasol ® 2002 | 10 | g |
| Extra D Nat Cos from Atofina) | | |
| Ethylene glycol dimethacrylate | 5 | g |
| and lauryl methacrylate | | |
| copolymer powder (Polytrap ® | | |
| 6603 Adsorber sold by RP | | |
| Scherer) | | |
| 0.9 Dtex polyamide fibers 0.3 | 5 | g |
| mm long (from Paul Bonte) | | |
| Pigments (iron oxides, | 10 | g |
| titanium dioxide) | | |

After mixing together the ingredients, the product was packaged in a dish by pressing. The product was easy to apply to the skin and allowed for uniform distribution of the makeup product on the skin.

### EXAMPLE 9

A skin matting product having the composition below was prepared:

| | | |
|---|---|---|
| Crosslinked polydimethylsiloxane | 40 | g |
| particles (DC 9506 from | | |
| Dow Corning) | | |
| Water | 19 | g |
| Glycerol | 5 | g |
| Propylene glycol | 5 | g |
| Preserving agents | 1 | g |
| Nylon powder (Orgasol ® 2002 | 20 | g |
| Extra D Nat Cos from Atofina) | | |
| Ethylene glycol dimethacrylate | 5 | g |
| and lauryl methacrylate | | |
| copolymer powder (Polytrap ® | | |
| 6603 Adsorber sold by RP | | |
| Scherer) | | |
| 0.9 Dtex polyamide fibers 0.3 | 5 | g |
| mm long (from Paul Bonte) | | |

After mixing together the ingredients, the product was packaged in a dish by pressing. The product was easy to apply to the skin.

1. A powdery composition comprising water, solid particles of elastomeric crosslinked organopolysiloxane and at least one additional particle, wherein the particles of elastomeric crosslinked organopolysiloxane and the at least one additional particle are present in an elastomeric crosslinked organopolysiloxane/additional particle weight ratio of greater than 1.2 and less than or equal to 2.5.

2. The composition of claim 1, wherein the crosslinked organopolysiloxane is chosen from those obtained:
via a crosslinking addition reaction of diorganosiloxane comprising at least one hydrogen linked to silicon and of diorganopolysiloxane comprising ethylenically unsaturated groups linked to silicon;
via a dehydrogenation crosslinking condensation reaction between a diorganopolysiloxane comprising hydroxyl end groups and a diorganopolysiloxane comprising at least one hydrogen linked to silicon;
via a crosslinking condensation reaction of a diorganopolysiloxane comprising hydroxyl end groups and of a hydrolysable organopolysilane;
via thermal crosslinking of organopolysiloxane; and
via crosslinking of organopolysiloxane by high-energy radiation.

3. The composition of claim 1, wherein the crosslinked organopolysiloxane is obtained via a crosslinking addition reaction (A) of diorganopolysiloxane comprising at least two hydrogens each linked to a silicon, and (B) of diorganopolysiloxane comprising at least two ethylenically unsaturated groups linked to silicon, in the presence (C) of a platinum catalyst.

4. The composition of claim 3, wherein the at least two ethylenically unsaturated groups are vinyl groups.

5. The composition of claim 1, wherein the crosslinked organopolysiloxane is obtained via reaction of dimethylpolysiloxane comprising dimethylvinylsiloxy end groups and of methylhydrogenopolysiloxane comprising trimethylsiloxy end groups, in the presence of a platinum catalyst.

6. The composition of claim 1, wherein the particles of elastomeric crosslinked organopolysiloxane are present in an amount ranging from 20% to 50% by weight, relative to the total weight of the composition.

7. The composition of claim 6, wherein the particles of elastomeric crosslinked organopolysiloxane are present in an amount ranging from 30% to 50% by weight, relative to the total weight of the composition.

8. The composition of claim 7, wherein the particles of elastomeric crosslinked organopolysiloxane are present in an amount ranging from 35% to 45% by weight, relative to the total weight of the composition.

9. The composition of claim 8, wherein the particles of elastomeric crosslinked organopolysiloxane are present in an amount ranging from 35% to 40% by weight, relative to the total weight of the composition.

10. The composition of claim 1, wherein the at least one additional particle is chosen from pulverulent dyestuffs, fillers, and fibers, and mixtures thereof.

11. The composition of claim 10, wherein the at least one additional particle comprises a pulverulent dyestuff.

12. The composition of claim 11, wherein the pulverulent dyestuff is chosen from pigments and nacres, and mixtures thereof.

13. The composition of claim 12, wherein the pulverulent dyestuff is chosen from titanium dioxide, zirconium oxides, cerium oxides, zinc oxides, iron oxides, chromium oxides, manganese violet, ultramarine blue, chromium hydrate, ferric blue, and metal powders.

14. The composition of claim 11, wherein the pulverulent dyestuff is present in an amount ranging from 0.5% to 50% by weight, relative to the total weight of the composition.

15. The composition of claim 14, wherein the pulverulent dyestuff is present in an amount ranging from 1% to 30% by weight, relative to the total weight of the composition.

16. The composition of claim 1, wherein the at least one additional particle comprises a filler.

17. The composition of claim 16, wherein the filler is chosen from talc, mica, silica, kaolin, polyamide powders, poly-β-alanine powders, polyethylene powders, tetrafluoroethylene polymer powders, lauroyllysine, starch, boron nitride, hollow microspheres of polyvinylidene chloride/acrylonitrile, hollow microspheres of acrylic acid copolymers, ethylene glycol dimethacrylate and lauryl methacrylate copolymer powders, silicone resin microbeads, precipitated calcium carbonate, magnesium carbonate, magnesium hydrocarbonate, hydroxyapatite, hollow silica microspheres, glass or ceramic microcapsules, metal soaps derived from organic carboxylic acids comprising from 8 to 22 carbon atoms, and mixtures thereof.

18. The composition of claim 17, wherein the filler is chosen from mica, polyamide powders, ethylene glycol dimethacrylate and lauryl methacrylate copolymer powders, boron nitride, and mixtures thereof.

19. The composition of claim 16, wherein the filler is present in an amount ranging from 0.5% to 40% by weight, relative to the total weight of the composition.

20. The composition of claim 19, wherein the filler is present in an amount ranging from 15% to 30% by weight, relative to the total weight of the composition.

21. The composition of claim 17, wherein the filler is present in an amount ranging from 1% to 35% by weight, relative to the total weight of the composition.

22. The composition of claim 21, wherein the filler is present in an amount ranging from 5% to 30% by weight by weight, relative to the total weight of the composition.

23. The composition of claim 1, wherein the additional particles comprise fibers.

24. The composition of claim 23, wherein the fibers are chosen from silk fiber, cotton fiber, wool fiber, flax fiber, cellulose fiber, rayon fiber, polyamide fiber, viscose fiber, acetate fiber, poly(p-phenyleneterephthalamide) fiber, acrylic fiber, polyolefin fiber, silica fiber, carbon fiber, polytetrafluoroethylene fiber, insoluble collagen fiber, polyester fiber, polyvinyl chloride fiber or polyvinylidene chloride fiber, polyvinyl alcohol fiber, polyacrylonitrile fiber, chitosan fiber, polyurethane fiber, polyethylene phthalate fiber, fibers formed from a mixture of polymers, substantially rectilinear rigid fibers, and mixtures thereof.

25. The composition of claim 24, wherein the fibers are chosen from rayon acetate fiber, polymethyl methacrylate fiber, poly(2-hydroxyethyl methacrylate) fiber, polyethylene fiber, polypropylene fiber, and carbon fiber in graphite form.

26. The composition of claim 23, wherein the fibers are fibers of synthetic origin.

27. The composition of claim 23, wherein the fibers have a length ranging from 1 μm to 10 mm.

28. The composition of claim 27, wherein the fibers have a length ranging from 0.1 mm to 5 mm.

29. The composition of claim 28, wherein the fibers have a length ranging from 0.3 to 1 mm.

30. The composition of claim 27, wherein the fibers have a cross section that is within a circle of diameter ranging from 2 nm to 500 μm.

31. The composition of claim 30, wherein the fibers have a cross section that is within a circle of diameter from 100 nm to 100 μm.

32. The composition of claim 31, wherein the fibers have a cross section that is within a circle of diameter from 1 μm to 50 μm.

33. The composition of claim 27, wherein the fibers have a length L and a diameter D such that L/D ranges from 3.5 to 2500.

34. The composition of claim 33, wherein L/D ranges from 5 to 500.

35. The composition of claim 34, wherein L/D ranges from 5 to 150.

36. The composition of claim 23, wherein the fibers are present in an amount ranging from 0.5% to 20% by weight, relative to the total weight of the composition.

37. The composition of claim 36, wherein the fibers are present in an amount ranging from 0.5% to 15% by weight, relative to the total weight of the composition.

38. The composition of claim 37, wherein the fibers are present in an amount ranging from 0.5% to 8% by weight, relative to the total weight of the composition.

39. The composition of claim 38, wherein the fibers are present in an amount ranging from 2% to 8% by weight, relative to the total weight of the composition.

40. The composition of claim 1, wherein the total amount of the at least one additional particle ranges from 20% to 50% by weight, relative to the total weight of the composition.

41. The composition of claim 40, wherein the total amount of the at least one additional particle ranges from 25% to 45% by weight, relative to the total weight of the composition.

42. The composition of claim 41, wherein the total amount of the at least one additional particle ranges from 25% to 40% by weight, relative to the total weight of the composition.

43. The composition of claim 42, wherein the total amount of the at least one additional particle ranges from 30% to 40% by weight, relative to the total weight of the composition.

44. The composition of claim 1, wherein the elastomeric crosslinked organopolysiloxane/additional particle weight ratio ranges from 1.21 to 2.

45. The composition of claim 44, wherein the elastomeric crosslinked organopolysiloxane/additional particle weight ratio ranges from 1.21 to 1.5.

46. The composition of claim 45, wherein the elastomeric crosslinked organopolysiloxane/additional particle weight ratio ranges from 1.21 to 1.3.

47. The composition of claim 1, wherein the water is present in an amount ranging from 5% to 30% by weight, relative to the total weight of the composition.

48. The composition of claim 47, wherein the water is present in an amount ranging from 10% to 25% by weight, relative to the total weight of the composition.

49. The composition of claim 48, wherein the water is present in an amount ranging from 15% to 25% by weight, relative to the total weight of the composition.

50. The composition of claim 49, wherein the water is present in an amount ranging from 20% to 25% by weight, relative to the total weight of the composition.

51. The composition of claim 1, further comprising a polyol.

52. The composition of claim 51, wherein the polyol comprises 2 to 20 carbon atoms.

53. The composition of claim 52, wherein the polyol comprises 2 to 10 carbon atoms.

54. The composition of claim 53, wherein the polyol comprises 2 to 6 carbon atoms.

55. The composition of claim 51, wherein the polyol is chosen from glycerol, propylene glycol, butylene glycol, pentylene glycol, hexylene glycol, dipropylene glycol, and diethylene glycol, and mixtures thereof.

56. The composition of claim 51, wherein the polyol is present in an amount ranging from 1% to 20% by weight, relative to the total weight of the composition.

57. The composition of claim 56, wherein the polyol is present in an amount ranging from 3% to 15% by weight, relative to the total weight of the composition.

58. The composition of claim 1, further comprising a cosmetic ingredient chosen from antioxidants, fragrances, preserving agents, neutralizers, surfactants, waxes, oils, sunscreens, vitamins, moisturizers, self-tanning compounds, and anti-wrinkle active agents.

59. The composition of claim 1, wherein the composition has a hardness ranging from 0.07 N to 0.4 N, and an elasticity ranging from 15% to 80%.

60. The composition of claim 59, wherein the composition has a hardness ranging from 0.1 to 0.35 N, and an elasticity ranging from 30% to 70%.

61. The composition of claim 1, wherein the composition is a cosmetic composition.

62. The composition of claim 61, wherein the composition is in the form of a free powder, a compact powder, a pressed powder, or a pasty composition.

63. The composition of claim 1, wherein the composition is a makeup or care composition for keratin materials.

64. The composition of claim 63, wherein the keratin material is the skin.

65. The composition of claim 63, wherein the makeup composition is a complexion product, an eyeshadow, an eyeliner, a makeup rouge, a concealer product, a body makeup product, a lip makeup product, a nail makeup product, or a hair makeup product.

66. The composition of claim 63, wherein the composition is a skin makeup composition.

67. The composition of claim 66, wherein the skin makeup composition is chosen from a complexion makeup composition and a foundation.

68. The composition of claim 66, wherein the skin makeup composition is a skincare composition chosen from a skincare product, a skin matting product, an anti-sun product for the skin, a self-tanning product, and a deodorant product.

69. A process for making up keratin materials comprising application of a composition comprising water, solid particles of elastomeric crosslinked organopolysiloxane and at least one additional particle, wherein the particles of elastomeric crosslinked organopolysiloxane and the at least one additional particle are present in an elastomeric crosslinked organopolysiloxane/additional particle weight ratio of greater than 1.2 and less than or equal to 2.5 to the keratin materials.

70. The process of claim 69, wherein the keratin material is the skin.

71. A method of making a makeup product that is easy to apply to a keratin material comprising adding water, solid particles of elastomeric crosslinked organopolysiloxane and at least one additional particle to the makeup product, wherein the particles of elastomeric crosslinked organopolysiloxane and the at least one additional particle are present in an elastomeric crosslinked organopolysiloxane/additional particle weight ratio of greater than 1.2 and less than or equal to 2.5.

72. The method of claim 71, wherein the makeup product, when applied to the keratin material, deposits uniformly on the keratin material.
